(19) United States
(12) Patent Application Publication
(10) Pub. No.: US 2024/0263119 A1
(43) Pub. Date: Aug. 8, 2024

(54) Cell culture vessel capable of obtaining a clean image by preventing distortion and dew condensation during cell imaging and method for producing same

(71) Applicant: CURIOSIS CO.,LTD, Seoul (KR)

(72) Inventors: Myeong Woo KANG, Yongin-si, Gyeonggi-do (KR); Tae Hwan SHIN, Icheon-si, Gyeonggi-do (KR); Yu Jin LEE, Yongin-si, Gyeonggi-do (KR); Sung Gyu SHIN, Seoul (KR); Ho Young YUN, Uijeongbu-si, Gyeonggido (KR)

(21) Appl. No.: 18/016,325

(22) PCT Filed: Jul. 27, 2022
(86) PCT No.: PCT/KR2022/011040

§ 371 Date: Jan. 13, 2023

(30) Foreign Application Priority Data
Jul. 22, 2022 (KR) 10-2022-0091158

(51) Int. Cl. C12M 1/00

(52) U.S. Cl. CPC C12M 23/22; C12M 23/20

(57) ABSTRACT

The present invention relates to a cell culture vessel. Specifically, the present invention relates to a cell culture vessel capable of obtaining a clean image by preventing occurrence of distortion and dew condensation when performing an imaging acquisition operation on cells cultured in the cell culture vessel.

### CROSS-REFERENCE TO RELATED APPLICATION

The present application is a national stage filing under 35 U.S.C § 371 of PCT application number PCT/KR2022/011040 filed on Jul. 27, 2022, which is based upon and claims the benefit of priority to Korean Patent Application No. 10-2022-0091158, filed on Jul. 22, 2022, in the Korean Intellectual Property Office. All of the aforementioned applications are incorporated herein by reference in their entireties.

### TECHNICAL FIELD

The present invention relates to a cell culture vessel. Specifically, the present invention relates to a cell culture vessel capable of obtaining a clean image by preventing occurrence of distortion and dew condensation when performing an imaging acquisition operation on cells cultured in the cell culture vessel.

### BACKGROUND ART

Recently, there is an increasing demand for live cell imaging equipment that can be utilized in cell experiments in the fields of biology, medical science, pharmacy, etc. Live cell imaging equipment can be utilized to establish a mechanism for how a drug can work in vivo to treat conditions and to conduct studies to determine efficacy and toxicity of a drug candidate in human. It can detect and analyze molecular reactions related to intracellular signal transduction and can be used to study the characteristics of stem cells. In particular, in a new drug development process, the live cell imaging can be effectively implemented for the new drug candidate screening by a method of long-term monitoring and quantitative analysis of the response from a cell line to a target drug.

Currently, commercialized instruments for the cell-based high content analysis (HCA) and the high content screening (HCS) mainly adopt methods for measuring various target molecules by fluorescence imaging in cells, in tissues, or in living body. These instruments mainly use laser scanning or spinning disc confocal microscopy, and can image various fluorescent labels with high resolution.

The cellular imaging is a process essentially carried out in most cell-based studies, and is performed with a cell culture vessel such as a petri dish, well plate, or flask for culturing cells being placed on a microscope.

In order to achieve the specific purposes such as checking the status of cell culture, checking cytotoxicity, and analyzing wound healing, it is important to obtain a clear and clean image for the accurate recognition of cell appearance, because the analysis is performed based on the imaged information obtained by cell imaging.

The problem is that, it is difficult to obtain an accurate image when a cell image is distorted by a meniscus phenomenon, which is a phenomenon in which the liquid surface of a culture solution in a cell culture vessel for culturing cells is crooked by surface tension.

The meniscus refers to the curved shape formed by the peripheral of the liquid surface in the capillary tube in contrast to the center of the surface due to surface tension. The shape of meniscus may be upward or downward, which is formed by the difference in the interaction between the liquids or the interaction between the liquid and the tube wall.

Specifically, it is a phenomenon caused by a contact angle between a solid and a liquid formed by surface tension. The contact angle refers to the angle formed by the interface of a liquid and a gas with a solid surface when the liquid and the gas are thermodynamically equilibrated on the solid surface.

The contact angle is often used as a measure to determine the wettability of a liquid with a particular solid. For example, a particular solid is expressed as hydrophilic when the contact angle with water is less than 90 degrees, and hydrophobic when it is greater than 90 degrees. A meniscus, which is a curved surface generated at the interface between the liquid in the container and the gas (atmosphere), is generated by this contact angle.

The meniscus is a purely natural phenomenon, but it causes problems in imaging the cells contained in the cell culture vessel. Most cellular images, in particular images of living cells, are produced via a bright field microscope.

Bright field imaging is an imaging method that observes changes in light passing through cells through an objective lens, thereby observing the appearance of cells through changes in light that is attenuated or refracted while passing through cells.

Thus, parallel light must be incident into the cells in order to obtain clear and clean cell images. However, as the meniscus formed on the surface of the culture solution in the cell culture vessel causes refraction of light, the light reaching the cells changes from parallel light into radiation or focused-beam type light, which distorts the image of the cells and consequently deteriorates the quality of the cell images, resulting in imbalance of focus, reduction in sharpness and contrast, etc. Since most cell culture vessels currently used are produced by hydrophilic treatment for the purpose of adhesion of cells, a meniscus in the form of a concave downward is inevitably formed when the culture medium is contained in such a hydrophilic-treated cell culture vessel.

Although Patent Document 1 attempts to physically remove the meniscus by pressing the wall surface of a container containing a cell culture solution, it has a disadvantage that a separate physical device is required to remove the meniscus.

In addition, since the characteristics of the cells may vary depending on the culture environment, humidity, temperature and acidity must be precisely maintained for survival and maintenance of cells during the imaging of living cells. Therefore, the imaging of living cells is most preferably performed inside a cell culture incubator.

However, since the cell culture incubator is under the high humidity environment, dew condensation occurs inside the cell culture vessel. Although the dew condensation is a natural phenomenon, it causes adverse issues in living cell imaging.

Live cell imaging is performed in a state where a cell culture vessel such as a petri dish, a well plate, or a flask for culturing cells is placed on a microscope. Live cell imaging equipment is often fabricated in an inverted microscope format. When imaging is performed by placing a cell culture vessel on top of the imaging equipment, the lower part of the cell culture vessel rises in temperature relative to the upper part due to the driving heat of the equipment. Therefore, the dew condensation occurs on the upper part of the cell culture vessel having a relatively low temperature. Since no thermal equilibrium occurs as long as the imaging equipment is driven, the dew condensation continues to grow. The dew condensation generated as such masks or induces refraction of the light irradiated from the top of the cell culture vessel for cell imaging. The cell image is thus reduced in brightness and blurred.

In order to overcome the dew condensation phenomenon occurring in cell culture vessels, various methods such as (1) heating the top of the cell culture vessel, (2) using a cell culture vessel with microchannel type, and (3) water-repellent coating have been tried.

Although these attempts can prevent the dew condensation to some extent, but they cause other problems such as temperature imbalance and limited applications.

The present invention intends to overcome meniscus phenomenon and the dew condensation phenomenon that may occur in cell imaging. If it is possible to acquire an undistorted image overcoming the meniscus issues and the dew condensation phenomenon, it is expected to be of great help in cell-related research.

### DISCLOSURE OF THE INVENTION

### Problems to be Solved by the Invention

An objective of the present invention is to provide a cell culture vessel in which distortion is prevented when performing an imaging acquisition operation on cells cultured in a cell culture vessel.

Another objective of the present invention is to provide a cell culture vessel in which the dew condensation does not occur during live cell imaging by coating a transparent material capable of absorbing the dew condensation in the cell culture vessel on the bottom surface of the cell culture vessel cover, and a method for producing the same.

The objectives of the present invention are not limited to those mentioned above, and other objectives and advantages of the present invention which are not mentioned can be understood by the following descriptions and will be more clearly understood by the examples of the present invention. It is readily apparent that the objectives and advantages of the present invention may be realized by the means and combinations indicated in the claims.

### Means for Solving the Problems

In order to solve the above problems, a cell culture vessel according to some embodiments of the present invention may comprise a culture solution receiving part having a bottom surface and side wall part to receive a cell culture solution (which corresponds to a “well” of “well plate” or a “container” of conventional cell cultures, and hereinafter is referred to as a “culture solution receiving part”); a transparent cover for covering the cell culture vessel body having the culture solution receiving part; wherein a hygroscopic polymer is provided on an inner lower surface of a transparent cover facing the cell culture vessel, thereby dew condensation is reduced inside the cell culture vessel and the clear images can be acquired.

The side wall part of the culture solution receiving part may comprise a first side wall part having a shape extending upward in a straight line from the edge of the bottom surface, and a second side wall part extending upward in a state inclined at a certain angle outward from the end of the first side wall part.

The angle of the second side wall part of the culture solution receiving part may include 20 to 50 degrees.

The side wall part of the culture solution receiving part may have an upward angular inclination outwardly from an edge of the bottom surface.

When the side wall part has a certain upward angular inclination outwardly from the edge of the bottom surface, the angle may include 20 to 50 degrees.

The cell culture vessel may comprise a water surface of the cell culture solution located on the second side wall part.

The cell culture vessel may comprise the hygroscopic polymer manufactured in the form of an adhesive type droplet absorption coating film and attached to the lower surface of the cell culture vessel cover, wherein the adhesive type droplet absorption coating film may be produced as a hygroscopic form capable of absorbing liquefied vapor or liquid material generated by the dew condensation.

The thickness of the hygroscopic polymer produced in the form of the adhesive film may be 1 um to 2 mm.

The cell culture vessel may comprise a hygroscopic polymer coated on the lower surface of the cell culture container cover.

The coating thickness of the hygroscopic polymer to be coated on the lower surface of the cover of the cell culture vessel may be 1 um to 2 mm.

An adhesive type droplet absorption coating film may be produced by attaching: a base film made of a transparent material; an adhesive applied to one side of the base film of the transparent material; a hygroscopic polymer coated on one side and the other side to which the adhesive is applied; and removable protective films attached to each of the adhesive-applied side and the hygroscopic polymer-coated side.

The adhesive type droplet absorption coating film may be attached to the lower surface of the cover of the cell culture vessel and used to suppress condensation inside the cell culture vessel.

The cell culture vessel coated with the hygroscopic polymer may be produced by a method of manufacturing a cell culture vessel comprising: a first step of coating a hygroscopic polymer on the lower surface of the cell culture vessel cover; and a second step of solidifying the coated hygroscopic polymer.

### Advantage of the Invention

The cell culture vessel according to the present invention overcomes a meniscus phenomenon that may occur during cell imaging, and suppresses the occurrence of the dew condensation generated by a temperature difference inside the cell culture vessel by coating or adhering hygroscopic polymer capable of absorbing liquefied vaper or liquid. In this way, it is possible to acquire a clear and clean image that can accurately grasp the appearance of cells. It has the effect of enabling accurate analysis based on the image information acquired by cell imaging from simple cell culture identification to special tasks such as cytotoxicity identification and wound healing analysis.

Further, the present invention provides an effect that a clear live cell image can be obtained by utilizing a conventional cell culture vessel, by way of attaching an adhesive film coated with a hygroscopic polymer capable of absorbing liquefied vapor or liquid generated by a temperature difference inside the cell culture vessel to the conventional cell culture vessel.

In addition to the above, the specific effects of the present invention will be described with reference to the following descriptions.

### BRIEF DESCRIPTION OF DRAWINGS

FIGS. 1A and 1B show a cell culture vessel according to the present invention.

FIGS. 2A and 2B are cross-sectional views of (A-A′) and (B-B′) of the cell culture vessel of FIGS. 1A and 1B, respectively, showing a plurality of culture solution receiving part.

FIGS. 3A and 3B are perspective views of an individual culture solution receiving part.

FIGS. 4A and 4B are photographs of actual cell culture showing the difference in cell images with or without meniscus formation in the cell culture vessel.

FIGS. 5A and 5B are conceptual diagrams for explaining the concept of the present invention.

FIG. 6 shows an individual culture solution receiving part embodying the principles of the present invention.

FIGS. 7A, 7B, 8A and 8B are experimental results showing the degree of meniscus generation in a typical cell culture vessel and in a cell culture vessel adopting an inclined structure to the culture solution receiving part, respectively.

FIG. 9 is a graph showing changes in contact angle with time when plasma surface treatment is performed on a culture vessel.

FIG. 10 is an example showing a typical cell culture vessel and problems due to the dew condensation that may occur during imaging of live cells using the same.

FIG. 11 is a schematic view of a case where a material absorbing dew condensation is coated on a lower surface of a cover to suppress occurrence of the dew condensation according to the present invention, and an enlarged view thereof.

FIG. 12 is a diagram showing an exemplary procedure of a method for producing a cell culture vessel according to an embodiment of the present invention, in which the lower surface of the cover of the cell culture vessel is activated with an aqueous KOH solution, and then a transparent material capable of absorbing liquid or vapor is coated to prevent condensation.

FIG. 13 is a diagram showing an exemplary procedure of a method for producing a cell culture vessel according to an embodiment of the present invention, in which the lower surface of the cover of the cell culture vessel is activated with a mixed solution of nitric acid and sulfuric acid, and then a transparent material capable of absorbing liquid or vapor is coated to prevent condensation.

FIG. 14 is a cross-sectional view of an adhesive film that can be attached to the lower surface of a cell culture vessel cover for use according to an embodiment of the present invention, and a method of using the same.

FIG. 15 is an exemplary flow chart of a method of manufacturing an adhesive film that can be attached to the lower surface of a conventional cell culture vessel cover for use according to an embodiment of the present invention.

FIG. 16 is a diagram showing the difference in occurrence of the dew condensation in a typical cell culture vessel and in a cell culture vessel in which a transparent material capable of absorbing liquid or vapor is coated on a lower surface of a cover of the cell culture vessel to prevent the dew condensation according to the present invention.

FIGS. 17A and 17B are diagrams showing the results of imaging for 24 hours in an incubator after a cell culture vessel in which a part of the lower surface of a cover is manufactured according to the method of the present invention is filled with water to which microbeads are added. FIG. 17A shows an unclear image due to the dew condensation of a portion not coated on the lower surface of the cover of the cell culture vessel, and FIG. 17B shows clearly photographed images obtained by a hygroscopic polymer coated on the lower surface of the cover.

### DESCRIPTION OF EMBODIMENTS

The terms or words used in the specification and claims should not be construed as being limited to their general or dictionary meanings. They should be interpreted as having a meaning and concept consistent with the spirit and scope of the invention in accordance with the principles that the inventors may define the concepts of terms or words in order to best explain their own invention. It should be understood that the embodiments described in the present specification and the configurations shown in the drawings are only one embodiment in which the present invention is realized, and do not represent all the technical ideas of the present invention, and thus, various equivalents, modifications, and applicable examples may be substituted at the time of filing the present application.

Although the terms such as first, second, A, B, etc. used in the specification and the claims may be used to describe various elements, these elements should not be limited by the above terms. These terms are only used to distinguish one element from another. For example, the first element could be termed the second element, and, similarly, the second elements could also be termed the first elements, without departing from the scope of the present invention. The term “and/or” includes a combination of a plurality of related items as listed or any of the plurality of associated items as listed.

The terminology used in the specification and the claims is for the purpose of describing particular embodiments only and is not intended to be limiting of the invention. The indefinite articles “a” and “an” include plural referents unless the context clearly dictates otherwise. In this application, terms such as “comprise” or “have” should be understood not to exclude the presence or addition of features, numbers, steps, operations, components, components or combinations thereof described in the specification.

In this specification and in the claims, when an element is referred to as being “connected” to another element, it should be understood to include the case where it is directly connected to the other element and the case when it is connected through another element in between. Only when the element is “directly connected” or “directly coupled”, it should be understood that one element and another element are connected without the other element in between. Similarly, other expressions describing a relationship between elements should be understood to be equivalent.

Unless otherwise defined, all terms used herein, including technical and scientific terms, have the same meaning as commonly understood by one of ordinary skill in the art to which this invention belongs.

Terms such as those defined in commonly used dictionaries should be interpreted as having a meaning that is consistent with their meaning in the context of the relevant art and will not be interpreted in an idealized or overly formal sense unless explicitly defined in this application.

In addition, each of configuration, procedure, process, or method included in each embodiment of the present invention may be shared to the extent not to be technically contradictory to each other.

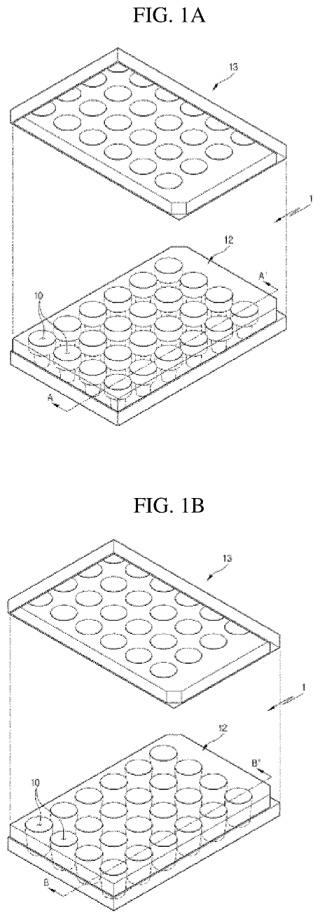

FIGS. 1A and 1B show a cell culture vessel according to the present invention.

The cell culture vessel 1 comprises a vessel body 12 having a culture solution receiving part 10 having a bottom surface and a tubular shape side wall part for receiving a cell culture solution, and a transparent cover 13 for covering the vessel body 1.

The lower surface of the cover 13 is coated with a hygroscopic polymer 14 to prevent the dew condensation.

Here, a part or all of the side wall part of the culture solution receiving part 10 is configured to be inclined outward from the edge of the bottom surface.

The tubular shape includes a cylindrical shape and an angular shape such as a rectangular cylindrical shape.

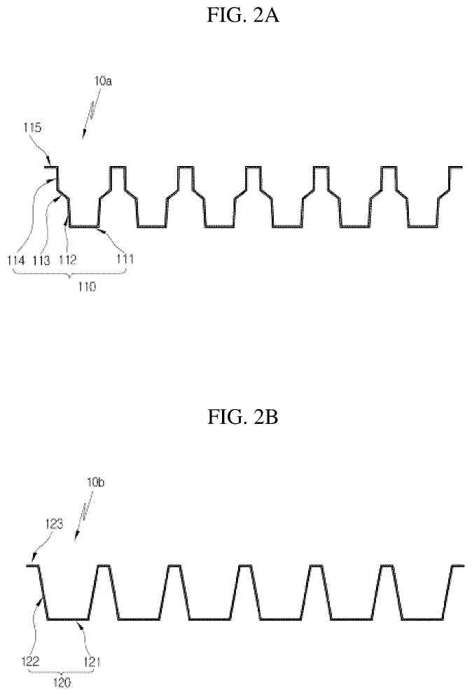

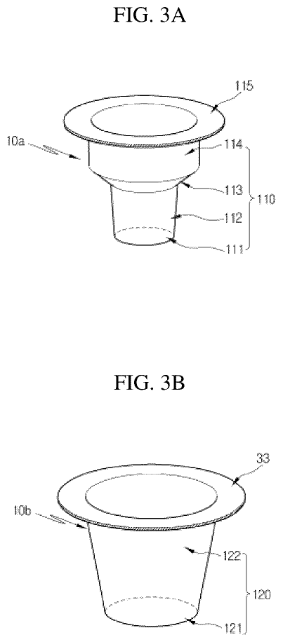

FIGS. 2A and 2B show cross-sections (A-A′) and (B-B′) of the cell culture vessel of FIGS. 1A and 1B, respectively, and FIGS. 3A and 3B show the individual culture solution receiving parts 10, 10a and 10b.

FIGS. 2A, 2B, 3A and 3B show different embodiments according to the form of the side wall part of the culture solution receiving part.

As shown in FIGS. 2A, 2B, 3A and 3B, in the cell culture vessel 1 according to the present invention, a part (FIG. 2A) or the whole (FIG. 2B) of the side wall part of the culture solution receiving part 10 is inclined outward with respect to the edges of the bottom surfaces 111,121.

On the other hand, even in the case of a typical culture vessel, it has an inclination gradient of about 1 to 5 degrees for extraction at the time of injection. The inclination in FIGS. 2B and 3B means that the inclination is given in addition to the inclination gradient in the typical cell culture vessel. In the present invention, “an inclined portion extending upward in a state of being inclined at a certain angle in the outward direction is formed on the side wall part” means that the meniscus phenomenon can be significantly reduced by having an inclination larger than the inclination gradient given for facilitating ejection at the time of injection.

As such, by inclining the side wall parts 110, 120 of the culture solution receiving part 10a, 10b, formation of a meniscus due to contact between the side wall part 110, 120 is suppressed, which will be described later.

As shown in FIGS. 2A and 2B, the vessel body 1 may be provided with a plurality of culture solution receiving parts 10,10a, and 10b for storing a cell culture solution.

FIGS. 3A and 3B show different embodiments of the culture solution receiving part.

First, as shown in FIG. 3A, each of the unit culture solution receiving part 10a extends upward from the bottom surface 111 of the culture solution receiving part 10a and is inclined outward from an arbitrary point, like a shape of a funnel.

The observation region may differ depending on the type of cells to be cultured or the timing to be monitored. Thus, the diameter of the bottom surface may be appropriately selected as necessary.

As shown in FIG. 3A, the funnel-shaped culture solution receiving part 10a comprises a bottom surface 111, a first side wall part 112 having a shape extending straight upward from an edge of the bottom surface 111, a second side wall part 113 extending upward in a state of being inclined at a certain angle outward from an end of the first side wall portion 112, a third side wall part 114 extending upward from the end of the second side wall part 113 and a connecting space 115 connecting the culture solution receiving parts 10a.

FIG. 3B shows another embodiment of the culture solution receiving part 10b of the present invention.

In this embodiment, the unit culture solution receiving part 10b is formed from the beginning by being inclined at a certain angle upward from the bottom surface 121 of the culture solution receiving part 10b.

As previously described, the inclination angle in this embodiment means that inclination is given in addition to the inclination gradient already given to a typical cell culture vessel.

The shapes of the culture solution receiving parts 10a, 10b shown in FIGS. 2A, 2B, 3A and 3B are only one example of the present invention. The shape is sufficient as long as it can suppress meniscus formed by contact between the side wall part and the culture solution due to an appropriate inclination provided to the side wall part of the culture solution receiving part.

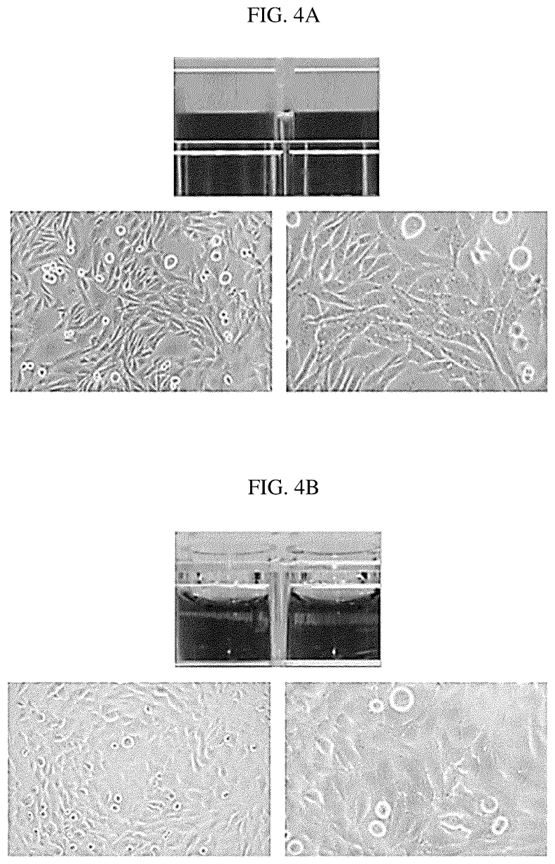

The followings will describe, with reference to FIGS. 4A to 9, the distortion phenomenon by the meniscus to be solved by the present invention and the feasibility of clear and accurate cell imaging when the formation of the meniscus is suppressed.

FIGS. 4A and 4B are real images showing differences in images obtained by imaging cells cultured in a cell culture vessel according to the presence or absence of meniscus formation in the cell culture vessel.

FIG. 4B shows a typical cell culture vessel. In the case of a typical cell culture vessel, the surface of the cell culture vessel is treated to be hydrophilic for cell adhesion, so that when a culture solution is filled in the cell culture vessel for cell culture, a meniscus of a downward concave shape is formed by surface tension as in the photo.

On the other hand, most live cell imaging equipment is manufactured as an inverted microscope type, which adopts a structure in which light is irradiated downward from a light source located on the upper part of the sample stage (the culture solution receiving part), and an image is captured with an objective lens from the lower part.

Therefore, when the meniscus is formed as shown in FIG. 4B, a lens effect is generated by the meniscus since the light incident from the upper part of the sample stage to the culture solution receiving part is refracted while passing through the culture solution, thereby the image of the cells to be imaged is distorted and unclear.

FIG. 4A is a cell culture vessel in which corona discharge coating is applied to the bottom part of a culture solution receiving part of the cell culture vessel in order to suppress formation of a meniscus. A discharge coating is applied to the bottom part so that the contact angle between the side wall of the cell culture vessel and the culture solution is close to a right angle. In this way, formation of a meniscus is suppressed, by which the surface of culture solution becomes flat, light is not refracted, and a clear image can be obtained without distortion.

However, as shown in FIG. 4A, performing the corona discharge coating on each of the bottom surface of the culture solution receiving part is not only difficult to process, but also requires an expensive treatment in terms of cost incurred. Therefore, the above setting is difficult to apply to typical cell cultures.

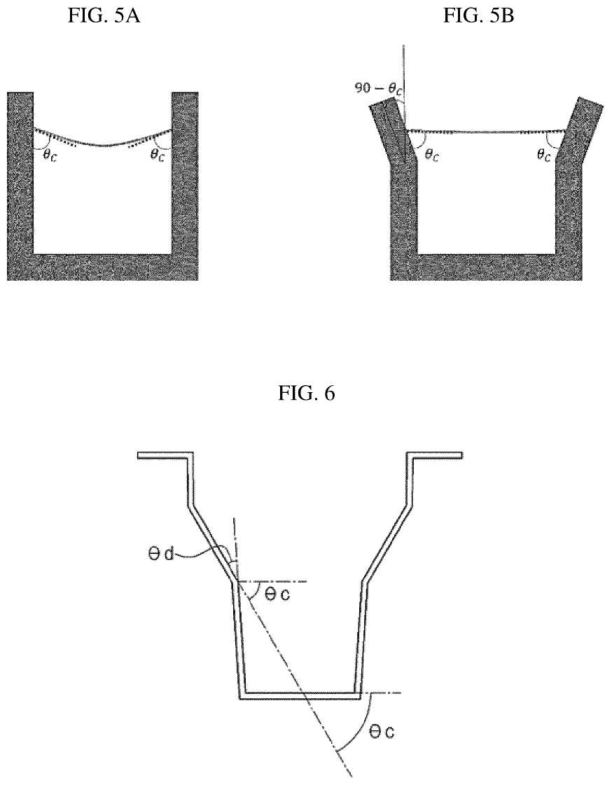

FIGS. 5A and 5B show the operational principle of the present invention. As shown in FIG. 5A, a concave meniscus is formed by the surface tension with the culture solution in the generally hydrophilic-treated culture solution receiving part

Here, when assuming that the contact angle (an acute angle) between the side wall of the culture solution receiving part and the surface of the culture solution is θc, the present inventors have found that the formation of a meniscus is suppressed when an inclination was applied to all or part of the side wall of the culture solution receiving part, as shown in FIG. 5B.

In addition, it was also found that the meniscus was most suppressed when the inclination angle was tilted outward by (90−θc) degrees (i.e., θd) from the side wall part of the culture solution receiving part.

Also, in the present invention, it was found that when θd is 20 to 50 degrees, the formation of meniscus was the smallest.

FIG. 6 shows an individual culture solution receiving part designed by applying the operational principles of the present invention described in FIGS. 5A and 5B.

In general, the cell culture vessel used for cell culture may be formed with a biocompatible polymer.

The biocompatible polymer may be any one of polystyrene (PS), polymethyl methacrylate (PMMA), cyclic olefin copolymer (COC), polycarbonate (PC), polytetrafluoroethylene (PTFE), polydimethylsiloxane (PDMS), polyvinylchloride (PVC), polyurethane (PU) and polyethylene terephthalate (PET).

The cell culture vessel made of such a biocompatible polymer may have some difference depending on the manufacturer, manufacturing specifications, etc. but the contact angle θc between the side wall of the cell culture vessel and the culture solution surface was about 40 to 70 degrees, and Od was given in consideration of this.

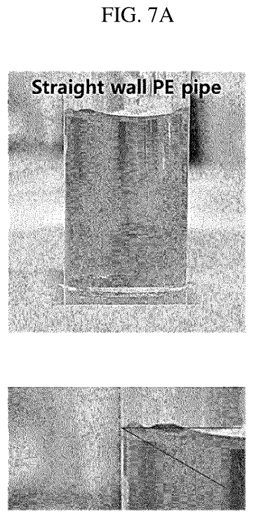

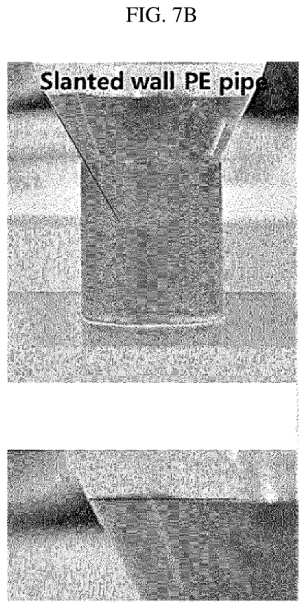

FIGS. 7A and 7B are experimental results on how a meniscus is formed in a conventionally used cell culture vessel (a general vessel in which a meniscus can be formed due to no or very small inclination at a side wall) and a cell culture vessel manufactured by arbitrarily inclining an upper part of a side wall of cell culture receiving part, respectively.

This experiment was conducted using a culture vessel manufactured by giving an inclination of 30 degrees as Od to the upper part of the side wall of the vessel part and a typical cell culture vessel with no inclination.

Both cell culture vessels were made of the same PE material, and the experiment was conducted under the same conditions except that an inclination was applied.

As shown in FIG. 7A, a meniscus was formed about 40 degrees with respect to the side of the vessel part of the typical cell culture vessel (the typical cell culture vessel with no inclination). In contrast, as shown in FIG. 7B, it was confirmed that the formation of meniscus was relatively small in the cell culture vessel with an inclination compared to the cell culture vessel in FIG. 7A, so that it retained an almost flat surface.

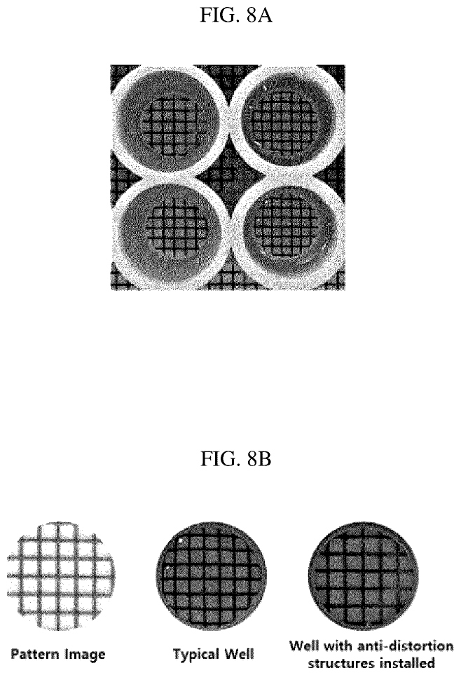

FIGS. 8A and 8B illustrate the results of photographing the pattern image from above each cell culture vessel part (FIG. 8A), after the pattern image made of grid paper is placed under the vessel part of the typical cell culture vessel and the cell culture vessel with an inclination formed on the side wall. As shown in FIG. 8A, in the case of the vessel part of cell culture vessel on the left with an inclination on the side wall of the cell culture vessel, an undistorted image can be obtained because meniscus is not generated. However, it is confirmed that occurred in the cell culture vessel part on the right, the image is distorted due to the generation of meniscus.

According to the present invention, when a light source irradiated above the cell culture vessel passes through the culture solution surface in order to obtain an image of cultured cells, one can clearly observe the shape of the cultured cells because the light is entered to the lens at the lower side of the cell culture vessel without refraction due to the lens effect. Of course, the same is true to a structure in which a light source is irradiated from lower side of the cell culture vessel and incident to the lens located above the cell culture vessel. the position of the light source and the lens of an imaging apparatus may be anywhere in the present invention, whose technical idea is that the phenomenon of refraction of the light source passing through the surface of a culture solution during cell imaging is reduced or restrained by suppression of meniscus.

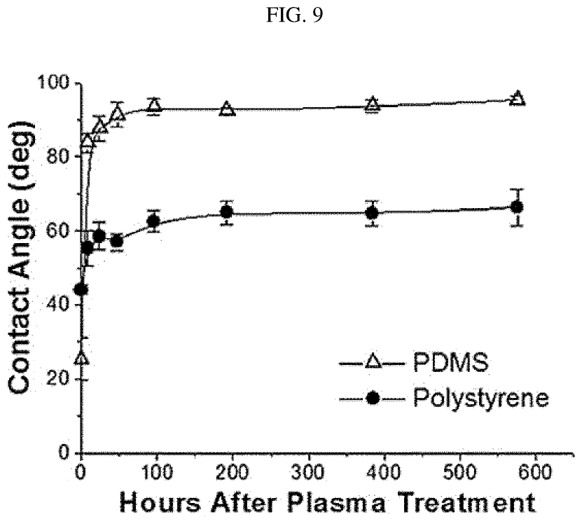

FIG. 9 is the experimental data for measuring how the contact angle between a side wall of a cell culture vessel and liquid changes with time after the hydrophilic surface treatment in a cell culture vessel composed of polystyrene and polydimethylsiloxane (PDMS), which are mainly used for the cell culture vessels.

In the case of polystyrene without hydrophilic surface treatment, the contact angle between the side wall of the cell culture vessel and the liquid is 90 degrees. On the other hand, as shown in FIG. 9, when the hydrophilic surface treatment (oxygen or nitrogen plasma treatment) is performed, the contact angle between the side wall of the cell culture vessel and the liquid instantaneously drops to around 40 degrees. And as time passes, the contact angle increases and converges to around about 60 degrees.

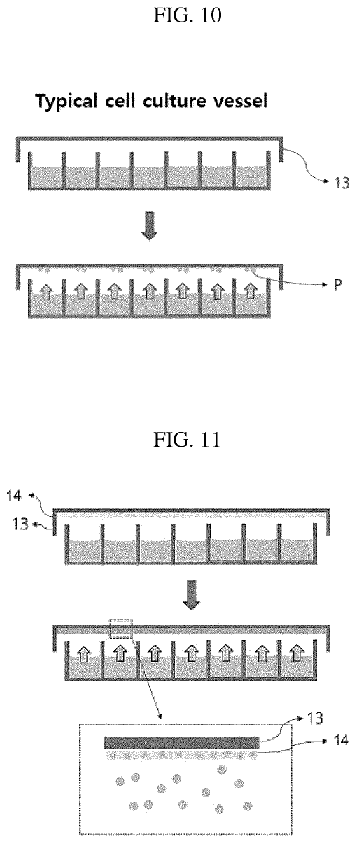

Accordingly, in the present invention, it is premised that a cell culture vessel is made of polystyrene. Regarding the inclination given to the side wall of the cell culture vessel and the inclination adopted by the inclined structure, the inclination (θd) of the side wall is given by assuming that the contact angle θc between the side wall of the cell culture vessel part and the culture solution is 40 to 70 degrees. FIG. 10 is a schematic of a typical cell culture vessel, which indicates that as the cell culture continues, the dew condensation(P) particles are attached to the lower surface of the cover 13 due to the high humidity environment. Since the cell culture incubator is a very high humid environment, the dew condensation is generally generated inside a cell culture vessel.

Hereinafter, with reference to FIGS. 11 to 17B, the operational principle on the cell culture vessel and the manufacturing method thereof to prevent the occurrence of the dew condensation, which is to be solved in the present invention, will be described as well as the feasibility of clear and accurate cell imaging when the occurrence of the dew condensation phenomenon is suppressed.

FIG. 11 is a schematic view of a case where a dew condensation absorbing substance 14 is coated on the lower surface of the cover 13 in order to suppress the occurrence of the dew condensation phenomenon according to the present invention.

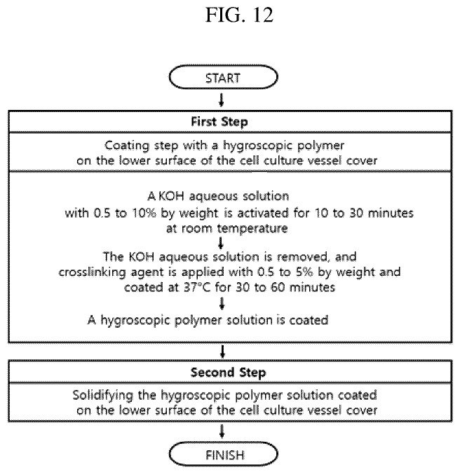

As shown in FIG. 12, when the lower surface of the cover 13 is coated with the hygroscopic polymer 14, the dew condensation that is occurred inside the cell culture vessel and formed on the lower side of the cover 13 is absorbed by the hygroscopic polymer 14, thereby the dew condensation on the cover 13 is prevented.

The cover 13 is made of a transparent material to obtain a clean and clear image when imaging live cells. In addition, it is sufficient if the cover 13 can be coated with a hygroscopic polymer 14 capable of absorbing liquefied vapor or liquid substances generated by the dew condensation. Preferably, it may be the cover of a culture dish, multi-well plate or the ceiling of the flask, but is not limited thereto.

The hygroscopic polymer 14 coated on the cover 13 may be preferably a hydrogel-based material such as collagen, fibrinogen, matrigel, gelatin, agarose gel, or the like. Alternatively, the hygroscopic polymer 14 coated on the cover 13 may be preferably a polymer-based material or a polymer material such as poly-vinyl alcohol (PVA), hydroxy-ethyl methacrylate (HEMA), poly-Vinyl pyrrolidone (PVP), poly (ethylene glycol) methacrylate) (PEGMA), Acrylamide, Polyacrylamide, Poly ethylene glycol (PEG), Sodium acrylate, Acrylic acid, Methacrylate, Sodium polyacrylate, glycerol acrylate, Diallyl carbonate, Methyl acrylate, ethyl acrylate, n-butyl acrylate, N-isopropyl acrylamide, poly N-isopropyl acrylamide, poly 2-(dimethylamino)ethyl methacrylate (PDMAEMA), Hydroxypropylcellulose (HPC), polyvinylcaprolactame, polyvinyl methyl ether, poly(N,N-dimethylacrylamide), Methacrylic acid-methyl methacrylate copolymer, methacrylic acid-methacrylate copolymer, poly(2-methoxyethyl acrylate) (PMEA), poly(oligo(ethylene glycol) (meth)acrylate) (POEG(M)A), poly(phosphobetaine methacrylate) (PPBMA), poly(sulfobetaine methacrylate) (PSBMA), poly(carboxybetaine) methacrylate (PCBMA), poly(serine methacrylate) (PSrMA), poly(2-alkoxyethyl acrylate), poly(2-alkoxyethyl vinyl ether), polylactides, polyglycolide, poly(ε-caprolactone), poly(trimethylene carbonate), poly(p-dioxanone), or the like. Alternatively, the hygroscopic polymer 14 coated on the cover 13 may be preferably a compound of the above polymer materials with a cross-linking agent such as poly(ethylene glycol) diacrylate (PEGDA), methylenebisacrylamide (MBA), Methacrylated alginate, Glycerol dimethacrylate, Methcrylated poly(succinimide), EGDMA (ethylene glycol dimethacrylate), N,N′-(1,2-dihydroxyethylene)bisacrylamide), 1,3-butanediol diacrylate, 1,6-hexanediol diacrylate, 1,4-butanediol diacrylate, glycerol 1,3-diglycerolate diacrylate, di(ethylene glycol) diacrylate, neopentyl glycol diacrylate, poly(propylene glycol) diacrylate, or the like. Alternatively, the hygroscopic polymer 14 coated on the cover 13 may be preferably a silicon-based material such as silica gel or silicone. However, as long as it is possible to obtain a clear image when imaging living cells by preventing the dew condensation from occurring, the hygroscopic polymer 14 is not limited to the substances listed above.

The cell culture vessel 1 may be manufactured by at least one method of catalytic reaction, thermal polymerization, ultraviolet polymerization, injection, heterogeneous injection, extrusion, printing or 3D printing, but is not limited thereto.

An initiator may be used in the method for manufacturing the cover 13. And the initiator may be used singly or in combination among the initiators such as ammonium persulfate, potassium persulfate, AIBN (Azobisisobutyronitrile), BPO (Benzoyl peroxide), Irgacure 2959 (2-hydroxy-4′-(2-hydroxyoxy)-2-methylpropionophen), Irgacure 651 (Benzyldimethyl ketal), Irgacure184 (1-hydroxy-cyclohexylphenyl ketone), Irgacure 819 (bisacrylphosphine oxides), Irgacure907 (2-Methyl-4′-(methylthio)-2-morpholinoproliophenone), sodium persulfate, sodium formaldehydesulfoxylate, tert-butyl hydroperoxide, 2-butanone peroxide, 2-hydroxy-2-methylpropiophenone, 3,4-dimethylbenzophenone, 3-hydroxybenzophenone, and etc. The initiator can be used without limitation as long as it can be dissolved in the dew condensation absorption solution, and preferably AIBN may be used.

The coating thickness of the hygroscopic polymer 14 is not particularly limited. However, when the coating thickness of the hygroscopic polymer 14 is too thin, it may be difficult to sufficiently absorb liquefied vapor or liquid substances generated by the dew condensation.

Considering that the present invention aims to perform live cell imaging without interference due to the dew condensation such as blocking or inducing refraction of light irradiated from upper part of the cell culture vessel, if the coating layer is too thick, a large amount of coating material is required, which is a disadvantage from the economic aspect.

Therefore, the thickness of the hygroscopic polymer 14 may be preferably between 1 um and 2 mm.

The coating area of the hygroscopic polymer 14 is not particularly limited. However, considering that the dew condensation may generally occur in the entire area or part of the lower surface of the cover of the cell culture vessel, the coating area of the hygroscopic polymer 14 may be equal to or smaller than the size of the inside of the cell culture vessel, but is not limited thereto.

The shape of the cell culture vessel 1 is not limited. Considering the purpose of preventing the formation of the dew condensation by coating the lower surface of the cover 13 with a hygroscopic polymer 14 capable of absorbing liquefied vapor or liquid substances due to the above-described condensation phenomenon, the cell culture vessel 1 may be any type of cell culture vessel used for cell culture that is generally made of a transparent material and can be coated with the hygroscopic polymer. Preferably, the shape of the cell culture vessel 1 may be a cell culture dish, multiplate, cell culture flask, or cell stack, but is not limited thereto.

In addition, since the material of the cell culture vessel 1 is not limited, the whole cell culture vessel or the whole cover or a part of the cover can be manufactured using the hygroscopic polymer 14 per se.

Since the cell culture vessel according to the present invention is for preventing the dew condensation from being generated during live cell imaging using the hygroscopic polymer 14 located on the lower surface of the cover 13 to obtain a clear image, it is not necessarily required to manufacture the cell culture vessel by coating the hygroscopic polymer from the time of production thereof. Also, the cell culture vessel according to the present invention includes the one in which an adhesive film is manufactured and attached to the lower surface of the cell culture vessel cover.

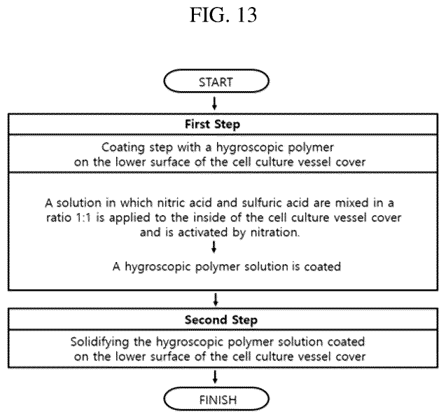

FIGS. 12 and 13 are diagrams showing a detailed process of manufacturing a cell culture vessel cover 13 according to the present invention. The cell culture vessel cover according to the present invention is manufactured by coating the lower surface of the cover 13 of the cell culture vessel 1 with a transparent hygroscopic polymer material 14 capable of absorbing the dew condensation and then solidifying it. Alternatively, a hygroscopic polymer made of a transparent material is manufactured in the form of an adhesive film 20 and attached to the lower surface of the cell culture vessel cover 13 immediately before use.

The method of manufacturing the cell culture vessel cover 13 according to the present invention may comprise a first step of coating a hygroscopic polymer 14 of a transparent material capable of absorbing liquefied vapor or liquid generated by the dew condensation on the lower surface of the cell culture vessel cover 13, and a second step of solidifying the coated hygroscopic polymer.

The first step of coating a hygroscopic polymer of a transparent material capable of absorbing liquefied vapor or liquid generated by the dew condensation on the lower surface of the cell culture vessel cover 13 consists of i) an activation step inside the cell culture vessel cover, ii) a crosslinking agent application and coating step for cross-linking the hygroscopic polymer with the inside of the cell culture vessel cover, and iii) a hygroscopic polymeric application step.

As a method for coating a hygroscopic polymer of a transparent material capable of absorbing the liquefied vapor or liquid generated by the dew condensation, there are mainly the following three methods: i) the plasma treatment on the lower surface of the cell culture vessel cover 13 and subsequent application of the hygroscopic polymer solution and polymerization. ii) the plasma treatment on the lower surface of the cell culture vessel cover 13 and subsequent coating of a pellet that can absorb the liquid or liquefied vapor generated by the dew condensation with the upper part at a high temperature and compression, iii) activating the surface of the lower surface of the cell culture vessel cover 13 and subsequent application of the hygroscopic polymer and polymerization. In this study, after activating the lower surface of the cell culture vessel cover 13, a hygroscopic polymer solution capable of absorbing liquefied vapor or liquid generated by the dew condensation was polymerized

The coating method includes a method of activating the lower surface of the cell culture vessel cover 13 with 0.5 to 10% by weight of an aqueous solution of potassium hydroxide (KOH) at room temperature for 10 to 30 minutes, or a method in which a solution in which nitric acid and sulfuric acid are mixed in a ratio 1:1 is applied and activated by the nitration.

After removing the hygroscopic polymer solution, a crosslinking agent capable of crosslinking the inside of the cell culture vessel cover and the hygroscopic polymer solution is applied with 0.5-5% by weight and coated at 37° C. for 30-60 minutes. The cross-linking agent may be a substance or a compound such as 3-(Trimethoxysilyl)propyl methacrylate, N-[3-(Trimethoxysilyl)propyl]ethylenediamine, 1-[3-(Trimethoxysilyl)propyl]urea, 3-(Trimethoxysilyl)propyl acrylate, (3-Glycidyloxypropyl)trimethoxysilane, 3-(Trimethoxysilyl)propyl methacrylate, (3-Mercaptopropyl)trimethoxysilane, (3-Aminopropyl)trimethoxysilane, (3-Triethoxysilyl-1-propanethiol), or the like.

The above process is a pre-treatment process for chemical bonding between the cell culture vessel cover 13 and the dew condensation absorbing solution. If the above process is omitted, the cell culture vessel cover 13 and the hygroscopic polymer 14 coating may be peeled off after the dew condensation is absorbed.

In the hygroscopic polymer 14 solution capable of absorbing liquefied vapor or liquid generated by the dew condensation, the polymer-based material has a function of absorbing condensation. In general, any compound capable of polymerizing and performing the function of a polymer chain structure may be used without limitation. Preferably PVP, PVA, or HEMA may be used in the polymer-based material. In addition, the crosslinking agent of the hygroscopic polymer solution capable of absorbing liquefied vapor or liquid generated by the dew condensation can be used without limitation as long as it is capable of crosslinking between compounds. Preferably, PEGDA may be used.

The hygroscopic polymer 14 solution consists of 50-90% by weight of HEMA, 10-50% by weight of PEGDA (Mn; 250), and 0.1-1% by weight of AIBN. The solution is not limited to the above range, and when the weight ratio of HEMA is low, i.e., when the weight ratio of PEGDA is high, the dew condensation absorption may be lowered. Conversely, the over-absorption of liquefied vapor or extra-liquid moisture generated by the dew condensation may affect live cell imaging.

In the second step of solidifying the hygroscopic polymer 14 solution coated inside the cell culture vessel cover 13, the hygroscopic polymer 14 solution is cured with 365 nm UV for 10 minutes, which may shorten the time in a higher output curing machine.

The inside of the coated cell culture vessel cover 13 absorbs and removes liquefied vapor or liquid that may be generated by the dew condensation during live cell imaging in the cell incubator.

The first step of coating the hygroscopic polymer 14 capable of absorbing liquefied vapor or liquid generated by the dew condensation of a transparent material inside the cell culture vessel cover 13 is conducted by the following steps: i) To activate the inside of the cell culture vessel cover, an aqueous KOH solution was applied to the inside of the cell culture vessel cover with 0.5 to 10% by weight and was activated for 10 to 30 minutes at room temperature; ii) the KOH aqueous solution was removed, rinsing was conducted with distilled water three times, and an aqueous solution of TMSPMA (3-(Trimethoxysilyl)propyl methacrylate) was applied with 0.5 to 5% by weight and coated at 37° C. for 30 to 60 minutes; and iii) After rinsing with distilled water three times, a hygroscopic polymer solution capable of absorbing liquefied vapor or liquid generated by the dew condensation was applied. In the second step, the hygroscopic polymer 14 solution coated on the lower surface of the cell culture vessel cover 13 was cured with 365 nm UV for 10 minutes.

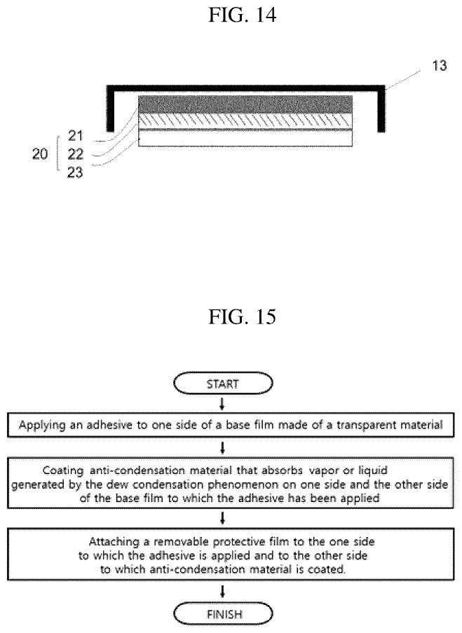

FIGS. 14 and 15 are diagrams of an adhesive film manufactured using a hygroscopic polymer 14 of a transparent material capable of absorbing liquefied vapor or liquid generated by the dew condensation phenomenon and a method for manufacturing the same.

The hygroscopic polymer 14 capable of absorbing liquefied vapor or liquid generated by the dew condensation phenomenon may be applied to an adhesive film, and the adhesive film may be used by attaching to a conventional cell culture vessel.

The method of manufacturing the adhesive film may comprise the following steps: i) applying an adhesive 21 to one side of a base film 22 made of a transparent material; ii) coating a hygroscopic polymer solution 23 that absorbs liquefied vapor or liquid generated by the dew condensation phenomenon on one side and the other side of the base film to which the adhesive has been applied; and iii) attaching a removable protective film to the one side of the base film to which the adhesive is applied and to the other side to which anti-condensation material is coated.

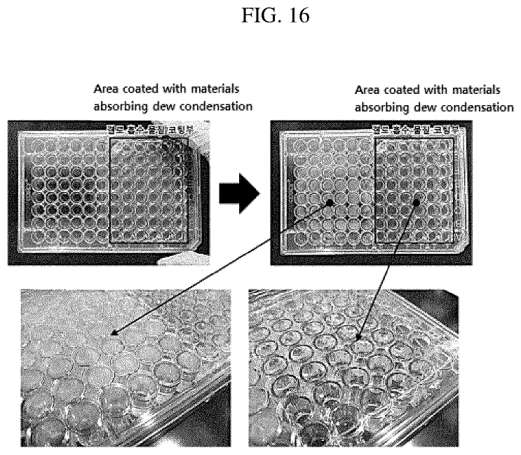

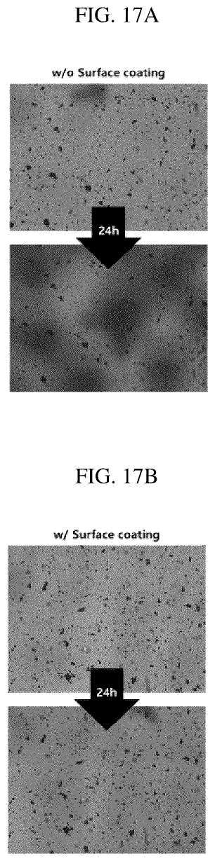

FIG. 16 shows an experimental result image that can visually confirm whether or not the dew condensation (P) occurs using the cell culture vessel 1 in which a part of the lower surface of the cell culture vessel cover 13 is coated with a condensation absorbing material. FIGS. 17A and 17B show images obtained by photographing live cells. As shown in FIGS. 16, 17A and 17B, it was found that the portion coated with the hygroscopic polymer 14 capable of absorbing the liquefied vapor or liquid generated by the dew condensation phenomenon does not generate the dew condensation (P). It was also found that a clear and bright image can be obtained when photographing live cells.

The above description is merely illustrative of the technical concept of the present embodiment, and a person skilled in the art to which this embodiment pertains may make various modifications and variations without departing from the essential characteristics of the present embodiment.

Accordingly, the present embodiments are intended to explain rather than limit the technical spirit of the present embodiment, and the scope of the technical spirit of the present embodiment is not limited by these embodiments.

The protection scope of the present embodiment should be interpreted by the following claims, and all technical ideas within the equivalent range should be interpreted as being included in the scope of the present embodiment.

1. A cell culture vessel capable of obtaining a clean image by preventing the dew condensation inside of the cell culture vessel, characterized by comprising a culture solution receiving part having a bottom surface and a side wall part to receive a cell culture solution; a transparent cover for covering the cell culture vessel body having the culture solution receiving part; wherein a hygroscopic polymer is provided on an inner lower surface of a transparent cover facing the cell culture vessel.

2. The cell culture vessel according to claim 1,
characterized in that a first side wall part having a shape extending upward in a straight line from the edge of the bottom surface, and a second side wall part extending upward in a state inclined at a certain angle outward from the end of the first side wall part.

3. The cell culture vessel according to claim 1,
characterized in that the side wall part has a certain angle upwardly from the edge of the bottom surface in an outward direction.

4. The cell culture vessel according to claim 2,
characterized in that the angle is 20 to 50 degrees.

5. The cell culture vessel according to claim 3,
characterized in that the angle is 20 to 50 degrees.

6. The cell culture vessel according to claim 2,
characterized in that a water surface of the cell culture solution. is located the second side wall part.

7. The cell culture vessel according to claim 1,
characterized in that the hygroscopic polymer is manufactured in the form of an adhesive type droplet absorption coating film and is attached to the lower surface of the cell culture vessel cover.

8. The cell culture vessel according to claim 7,
characterized in that a coating thickness of the hygroscopic polymer manufactured in the form of the adhesive film is 1 um to 2 mm.

9. The cell culture vessel according to claim 1, characterized in that the hygroscopic polymer is coated on a lower surface of a cover of the cell culture vessel.

10. A cell culture vessel according to claim 9,
characterized in that the coating thickness of the hygroscopic polymer coated on the lower surface of the cover of the cell culture vessel is 1 um to 2 mm.

11. (canceled)

12. (canceled)

13. (canceled)

14. A method of manufacturing a cell culture vessel comprising a first step of coating a hygroscopic polymer on the lower surface of the cell culture vessel cover; and a second step of solidifying the coated hygroscopic polymer.
